(19) United States
(12) Patent Application Publication
(10) Pub. No.: US 2021/0161720 A1
(43) Pub. Date: Jun. 3, 2021

(54) MEDICAL DRESSING REMOVABLE ADHESIVE STRIPS

(71) Applicant: CARDIAC ASSIST HOLDINGS, LLC, Ann Arbor, MI (US)

(72) Inventors: Allen B. Kantrowitz, Ann Arbor, MI (US); Barry N. Gellman, Ann Arbor, MI (US); Kurt A. Dasse, Ann Arbor, MI (US)

(73) Assignee: CARDIAC ASSIST HOLDINGS, Ann Arbor, MI (US)

(21) Appl. No.: 17/045,243

(22) PCT Filed: Apr. 4, 2019
(86) PCT No.: PCT/US2019/025738

§ 371 Date: Oct. 5, 2020

Related U.S. Application Data
(60) Provisional application No. 62/652,395, filed on Apr. 4, 2018.

(51) Int. Cl. A61F 13/00; A61F 13/02; A61M 5/142; A61M 60/148; A61M 60/871

(52) U.S. Cl. CPC A61F 13/00051; A61F 13/0226; A61F 13/025; A61M 2005/1416; A61M 60/148; A61M 60/871; A61M 5/14248

(57) ABSTRACT

Medical dressings are provided that minimize the disruptive forces directed at the device-skin interface during the processes of dressing changes. The instantaneous disruptive force, imparted to a healing skin wound by an adhesive dressing as it is being de-adhesed from the vicinity of the skin wound, is determined, in part, by the yield strength (force/unit area) of the adhesive/skin interface and, in part, by that portion of surface area (area) of skin-dressing adhesion participating in traction of the skin at said instant. A method to minimize the disruptive force of a medical dressing is to reduce the surface area of skin-dressing adhesion being de-adhesed at a specific instant by dividing the total surface area of skin-dressing into substantially smaller subareas, each of which, when being de-adhesed, would impart disruptive forces to the healing skin wound which are smaller than the tensile strength of the skin wound.

### RELATED APPLICATIONS

This application claims priority benefit of U.S. Provisional Application Ser. No. 62/652,395 filed 4 Apr. 2018, the contents of which are hereby incorporated by reference.

### FIELD OF THE INVENTION

The present invention in general relates to medical devices and systems and in particular to removable adhesive strips and patches for post-operative securement of a percutaneous access device (PAD) or other medical devices following implantation and during tissue growth.

### BACKGROUND OF THE INVENTION

Heart disease is one of the leading causes of death. Currently, medical science cannot reverse the damage done to the cardiac muscle by heart disease. One solution for such patients is a heart transplant. However, the number of cardiac patients in need of a heart transplant far exceeds the limited supply of donor hearts available.

The scarcity of human hearts available for transplant, as well as the logistics necessary to undertake heart transplant surgery, makes an implantable cardiac assist device a viable option for many heart patients. A blood pump can be surgically implanted in, or adjacent to the cardiovascular system to augment the pumping action of the heart. The blood pump is sometimes referred to as a mechanical auxiliary ventricle assist device, dynamic aortic patch, balloon pump, mechanical circulatory assist device, or a total mechanical heart. Alternatively, the blood pump can be inserted endovascularly.

Typically, the blood pump systems include a driveline that serves as a power and/or signal conduit between the blood pump internal to the patient and a controller/console external to the patient.

Often a percutaneous access device (PAD) can be surgically implanted body at the location in the skin where the driveline penetrates the skin to provide a through-the-skin coupling for connecting the supply tube to an extra-corporeal fluid pressure source. Alternatively, the fluid pressure source can be implanted wholly within the body, energized by electromagnetic means across intact skin, or energized by or chemical energy found within the body or some other means. Electrical leads from electrodes implanted in the myocardium are likewise brought out through the skin by means of the PAD. The aortic valve status or any cardiovascular parameter that is associated with this status can be employed to control the fluid pressure source to inflate and deflate the inflatable chamber in a predetermined synchronous relationship with the heart action.

The surface of the driveline, or of the optional PAD used in cardiac assist system may have characteristics which promote the formation of a natural biologic seal between the skin and the device to form a barrier to microbial invasion into the body at the skin penetration site. Percutaneous access devices may also illustratively be used for other devices including peritoneal dialysis catheters, Steinman pin, Kirschner wires, and chronic indwelling venous access catheters that require skin penetration. More generally, medical appliances which are implanted so as to cross the skin surface and therefore violate the “barrier function” of the skin, may also illustratively be used for other medical purposes including peritoneal dialysis catheters and, chronic indwelling venous access catheters, neurologic prostheses, osseointegrated prostheses, drug pumps, and other treatments that require skin penetration.

FIG. 1 illustrates wearable and implanted components of an exemplary prior art cardiac assist system. A PAD 10 serves as an attachment point for an external supply line 12 that supplies air or fluid from a wearable external drive unit (EDU) 14. The EDU 14 is powered by a wearable battery pack 16. Inside the body of the patient, a drive line 18 is attached to the PAD 10 and provides an air or fluid conduit to a cardiac assist device 20.

A common problem associated with implantation of a PAD or other skin penetrating appliance is skin regeneration about the periphery of the appliance to form an immunoprotective seal against infection. New cell growth and maintenance is typically frustrated by the considerable mechanical forces exerted on the interfacial layer of cells. In order to facilitate skin regeneration about the exterior of the appliance, subject cells are often harvested and grown in culture onto appliance surfaces for several days prior to implantation in order to allow an interfacial cell layer to colonize appliance surfaces in advance of implantation. Unfortunately, cell culturing has met with limited acceptance owing to the need for a cell harvesting surgical procedure preceding the implantation procedure. Additionally, maintaining tissue culture integrity is also a complex and time-consuming task.

A related context in which cell growth is needed is wound healing, with DACRON® based random felt meshes have been used to promote cell regrowth in the vicinity of a wound, such felts have uncontrolled pore sizes that harbor bacterial growth pockets.

U.S. Pat. No. 7,704,225 to Kantrowitz solves many of these aforementioned problems by providing cell channeling contours, porous biodegradable polymers and the application of vacuum to promote cellular growth towards the surface the neck of a PAD. The facilitating of rapid cellular colonization of a PAD neck allows the subject to act as their own cell culture facility and as such affords more rapid stabilization of the PAD, and lower incidence of separation and infection.

FIG. 2 depicts a PAD generally at 100 as shown in U.S. application Ser. No. 13/416,546 to Kantrowitz. A cap 102 is formed of a material such as silicone, a polymer or a metal and serves to keep debris from entering the device 100. Preferably, the cap 102 is remote from the surface of the epidermis E. The medical appliance 34 depicted as a catheter and vacuum or hydrodynamic draw tubing 104 pass through complementary openings 106 and 108, respectively formed in the cap 102. The tubing 104 provides fluid communication between a vacuum or hydrodynamic draw source 22 and an inner sleeve 13. The inner sleeve 13 is characterized by a large and rigid pore matrix 19 in fluid communication to a vacuum source 22 such that the source 22 draws (arrow 22D) tissue fluid and fibroblasts 21 into the sleeve 13 Sleeve 13 has a surface 24 that is optionally nanotextured to promote fibroblast adhesion. The surface 24 is optionally decorated with a pattern of contoured cell-conveying channels. It is appreciated that inner sleeve 13 optionally includes matrix 26 thereover, a coating substance 27, or a combination thereof. The coating 27 is appreciated to need not cover the entire surface 24. The tissue contacting surface 29 of substance 27 is optionally nanotextured. A flange 112 is provided to stabilize the implanted device 100 within the subcuteanous layer S. The flange 112 is constructed from materials and formed by methods conventional to the art. For example, those detailed in U.S. Pat. Nos. 4,634,422; 4,668,222; 5,059,186; 5,120,313; 5,250,025; 5,814,058; 5,997,524; and 6,503,228.

FIGS. 3A-3C illustrate a modular external interface housing 200 coupled to the PAD 100 as disclosed in U.S. application Ser. No. 15/555,952 to Subilski. The modular external interface 200 forms a collar about the neck 110 of the PAD 100 with the main body 216 with a locking feature 218, such as a male extension that engages a female receptacle or cavity as a mechanical overlap connection. In a specific embodiment the main body 216 is made of silicone. The collar seal between the main body 216 and the neck 110 of the PAD 100 forms a hermetic seal with a gasket 230, which in a specific embodiment is a flexible gasket integrated into the main body 216. In a specific embodiment the gasket 230 may be a floating gasket. The stabilization of the PAD 100 within the skin to form a germ-free barrier requires subject cells to grow onto the neck surfaces 17 as shown in FIG. 2 of the PAD 100 adjacent to the subject's epidermis E. The neck surface region 17 is adapted to promote growth of autologous fibroblast cells thereon. A suitable exterior side surface substrate for fibroblast growth is a nanotextured polycarbonate (LEXAN®). The modular external interface 200 has a central opening 220 adapted at least one drive line for insertion into a PAD, and a portal 224 for a vacuum line 222.

The modular external interface 200 is secured and sealed to an outer layer of a patient's skin with a medical dressing. In a specific embodiment the medical dressing is a preform patterned and shaped to conform to the exterior of the modular external interface 200. In a specific embodiment the medical dressing preform may be in two halves (212 214) that overlap. In a specific embodiment the medical dressing preform may be transparent. In a specific embodiment the medical dressing preform may be made of Tegaderm™ manufactured by Minnesota Mining and Manufacturing Company.

Despite the advances in PAD design and the securement of PAD to a subject's skin there continues to be a problem of disrupting the formation of skin layers about the PAD with the removal of medical dressings during dressing changes during the healing process.

There is a continuing need for improved medical dressings that minimize the disruptive forces to nascent layers of skin that are being formed during the healing process

### SUMMARY OF THE INVENTION

Medical dressings are provided that minimize the disruptive forces directed at the device-skin interface during the processes of dressing changes. The instantaneous disruptive force, imparted to a healing skin wound by an adhesive dressing as it is being de-adhesed from the vicinity of the skin wound, is determined, in part, by the yield strength (force/unit area) of the adhesive/skin interface and, in part, by that portion of surface area (area) of skin-dressing adhesion participating in traction of the skin at said instant.

A method to minimize the disruptive force of a medical dressing is to reduce the surface area of skin-dressing adhesion being de-adhesed at a specific instant by dividing the total surface area of skin-dressing into substantially smaller subareas, each of which, when being de-adhesed, would impart disruptive forces to the healing skin wound which are smaller than the tensile strength of the skin wound.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIG. 1 illustrates prior art wearable and implanted components of a cardiac assist system with a percutaneous access device (PAD) and internal driveline;

FIG. 2 is a prior art, partial cutaway view of a flanged percutaneous access device (PAD) with relative dimensions of aspect exaggerated for visual clarity;

FIGS. 3A-3C are perspective views of a prior art modular external interface seal for a PAD appliance secured with adhesive dressings to a subject;

FIG. 4A illustrates a coiled medical dressing in accordance with an embodiment of the invention;

FIG. 4B illustrates a serpentine medical dressing in accordance with an embodiment of the invention;

FIG. 4C illustrates a “split-ring” style medical dressing in accordance with an embodiment of the invention;

FIGS. 5A-5C illustrate various patterns of adhesive backings for rectangular dressings for minimizing disruptive forces to a wound area in accordance with embodiments of the invention; and

FIG. 6 is a bottom perspective view of a center perforated medical dressing in accordance with an embodiment of the invention.

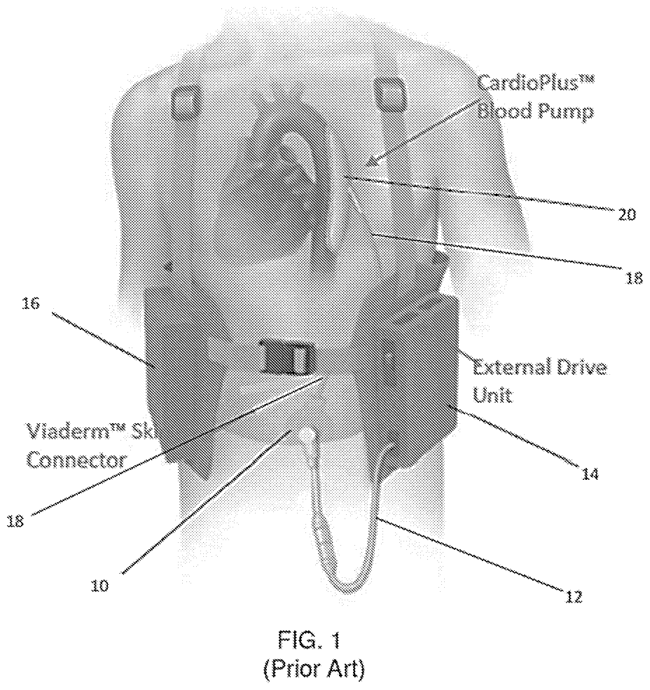

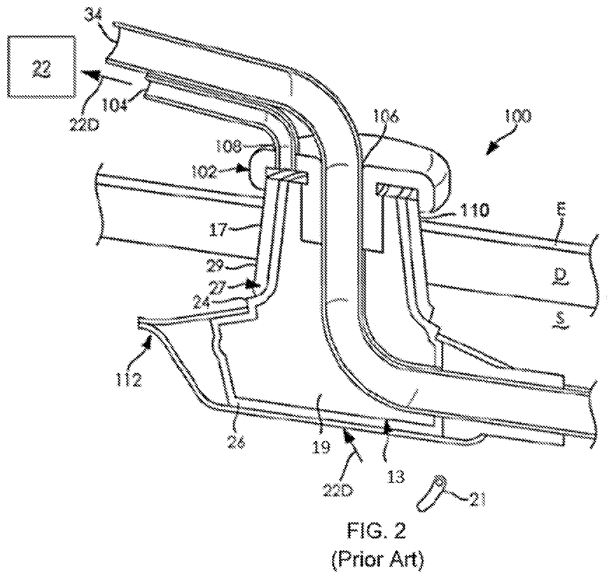

### DETAILED DESCRIPTION OF THE INVENTION

Medical dressings according to the present invention have utility to significantly minimize the disruptive forces directed at the device-skin interface during the processes of dressing changes. It is appreciated that device interfaces include percutaneous access devices (PAD). PAD used herein may include PAD used in cardiac assist systems that promote the formation of a natural biologic seal between the skin and the device to form a barrier to microbial invasion into the body. Percutaneous access devices may also illustratively be used for other devices including peritoneal dialysis catheters, Steinman pin, Kirschner wires, and chronic indwelling venous access catheters that require skin penetration.

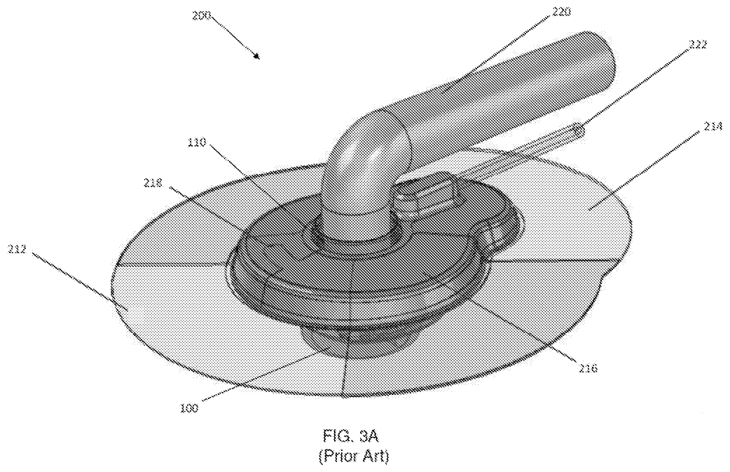

The instantaneous disruptive force, imparted to a healing skin wound by an adhesive dressing as it is being de-adhesed from the vicinity of said skin wound, is determined, in part, by the yield strength (force/unit area) of the adhesive/skin interface and, in part, by that portion of surface area (area) of skin-dressing adhesion participating in traction of the skin at said instant. Ideally, the instantaneous disruptive force imparted to a healing skin wound is less than the then-extant skin wound tensile strength. A useful means to reduce the disruptive force created by removal of a dressing is to reduce the portion of surface area of skin-dressing adhesion being separated at any specified instant. A means to accomplish a reduction of surface area of skin-dressing adhesion being de-adhesed at a specific instant is to divide the total surface area of skin-dressing into substantially smaller subareas, each of which, when being de-adhesed, would impart disruptive forces to the healing skin wound which are smaller than the tensile strength of the skin wound. Herein we refer to such dressings a “low-detachment force” dressings. Embodiments of the inventive medical dressings may include peel-away dressings manufactured with a serpentine pathway which limits the skin-adhesive contact area which is being pulled apart at any one moment as the dressing is being removed from the skin. In an inventive embodiment, a coiled “Boa” style could be used for general medical applications currently treated with a rectangular adhesive dressing such as a Tegaderm™ or similar product. In a specific inventive embodiment, a serpentine “Sidewinder” style could also be used for rectangular dressings or could be manufactured in “roll tape” form. An inventive “split-ring”, or alternatively a paired hemi-dressing, style is provided for ViaDerm™ implementations as shown in FIGS. 3A-3C or could also be used for any “drain dressing” or “medical appliance” dressing.

Embodiments of the inventive medical adhesive dressings may be applied conventionally. At the time of removal, the serpentine pathway may be activated for the removal maneuver by pulling a separation thread, removing a secondary backing layer, pulling against premanufactured perforation pathways, or other more active measures illustratively including a special removal tool, or photo-activated detach sites.

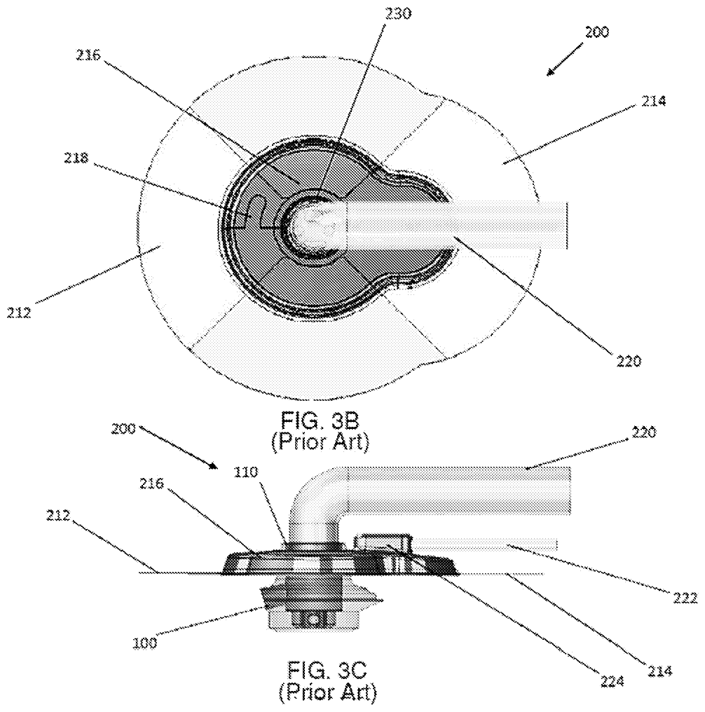

Referring now to the figures, FIG. 4A illustrates a coiled “boa style” medical dressing 300 with a separation thread 302. Embodiments of the coiled medical dressing 300 may utilize a low-detachment force adhesive dressing and is suitable for pre-manufactured rectangular-type dressings. FIG. 4B illustrates a serpentine “sidewinder style” medical dressing 310. Embodiments of the serpentine medical dressing 310 may utilize a low-detachment dressing force adhesive and is suitable for pre-manufactured rectangular-type dressings or a roll tape style dispenser. FIG. 4C illustrates a “split-ring” style medical dressing 320 for use with the ViaDerm™ implementation as shown in FIGS. 3A-3C or could also be used for any “drain dressing” or “medical appliance” dressing. As the medical dressing 320 is pulled up during a dressing change, the resultant pull force on the wound area alternates in direction as sections of the dressing 320 are lifted up. The dressing 320 has a channel 322 to fit around features of a medical appliance or PAD to be secured, and a cutout 324 to accommodate a medical device or neck of a PAD to be secured. Embodiments of the “split-ring” style medical dressing 310 may utilize a low-detachment force adhesive dressings.

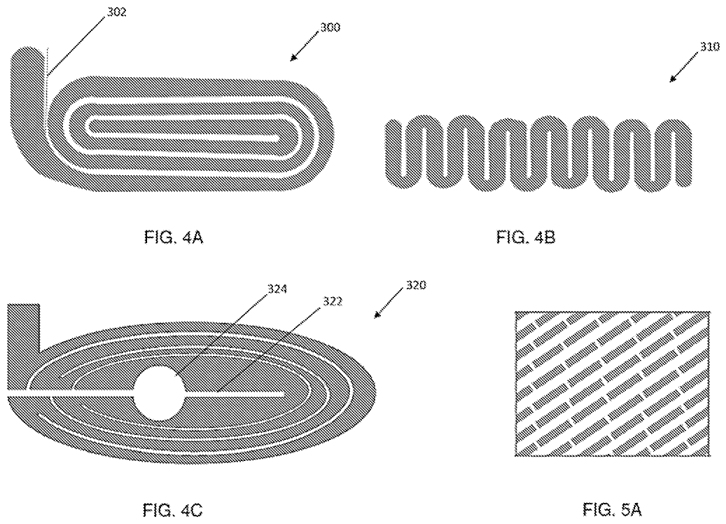

FIGS. 5A-5C illustrate various patterns of low-detachment force adhesive backings for rectangular or other shaped dressings for minimizing disruptive forces to a wound or surgical device area. The exemplary patterns of adhesive have breaks or changes in direction to minimize or control the forces exerted on the skin or device being secured with the medical dressing as the dressing is being lifted for removal. It is appreciated that additional adhesive patterns and shapes may also be used to minimize or control the forces exerted on the skin or device during removal.

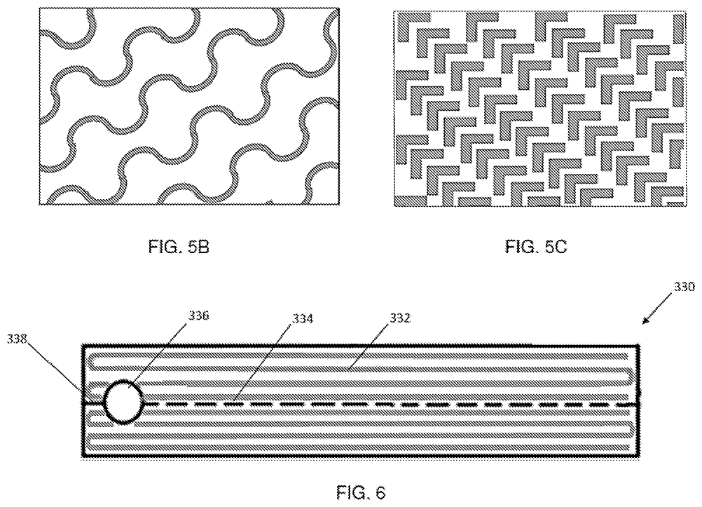

FIG. 6 is a bottom perspective view of a center perforated medical dressing 330. The center perforation 334 allows the medical dressing 330 to be removed in sections. A slit 338 at one end of the dressing allows a medical device or neck of a PAD to be placed in the cutout 336.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A medical dressing that minimizes disruptive forces at a device-skin interface during a dressing change, said medical dressing comprising:
a serpentine, coiled, or discontinuous pattern of an adhesive applied to the contact surface of the medical dressing.

2. The medical dressing of claim 1 wherein the adhesive is a low-detachment force adhesive.

3. The medical dressing of claim 1 further comprising a channel to fit around features of a medical appliance or percutaneous access devices (PAD) to be secured, and a cutout to accommodate a medical device or neck of a PAD to be secured.

4. The medical dressing of claim 1 further comprising at least one of a separation thread, a secondary backing layer, or perforation pathways.

5. The medical dressing of claim 1 further comprising photo-activated detach sites.

6. The medical dressing of claim 1 wherein the medical dressing is pre-manufactured as a rectangular-type dressing or in a roll tape.
